(19) United States
(12) Patent Application Publication
(10) Pub. No.: US 2025/0290839 A1
(43) Pub. Date: Sep. 18, 2025

(54) METHOD AND SYSTEM FOR DETECTING FIRES VIA LIGHT DETECTION AND RANGING (LIDAR) AND/OR TIME-OF-FLIGHT (TOF) SENSORS

(71) Applicant: Mobile Physics Ltd., Kfar Saba (IL)

(72) Inventor: Erez WEINROTH, Kfar Saba (IL)

(73) Assignee: Mobile Physics Ltd., Kfar Saba (IL)

(21) Appl. No.: 19/222,164

(22) Filed: May 29, 2025

Related U.S. Application Data
(63) Continuation of application No. PCT/IL2023/051231, filed on Nov. 30, 2023.
(60) Provisional application No. 63/429,524, filed on Dec. 1, 2022.
(60) Provisional application No. 63/482,614, filed on Feb. 1, 2023.

(51) Int. Cl. G01N 15/075; G01N 15/00; G01S 17/88

(52) U.S. Cl. CPC G01N 15/075; G01S 17/88; G01N 2015/0046

(57) ABSTRACT

Method and system for early detection of an indoor fire using either a smartphone or a standalone device having a light detection and ranging (LiDAR) sensor or Time of Flight (TOF) sensor, are provided herein. The system may include: a processing device; a memory device installed on the mobile communication device storing a set of instructions that, when executed, cause the processing device to: obtain reflection measurements from the LiDAR/TOF sensor, wherein reflections are measured for a plurality of segments along a line of sight of the LiDAR/TOF sensor; calculate a level of air particles in an ambience of the mobile communication device, based on the reflection measurements, and further based on calibration data model which maps the reflection measurements onto level of air particles; and detect smoke within the ambience of the mobile communication device, whenever the calculated level of air particles goes beyond a predefined threshold.

### CROSS REFERENCE TO RELATED APPLICATION

This application is a continuation of PCT Application No. PCT/IL2023/051231, International Filing Date Nov. 30, 2023, which claims the benefit of U.S. Provisional Patent Applications Nos. 63/429,524, filed on Dec. 1, 2022 and 63/482,614, filed on Feb. 1, 2023, all of which are incorporated herein by reference in their entireties.

### FIELD OF THE INVENTION

The present invention relates generally to the field of detecting fires, and more particularly to detecting fires based on signals sensed by sensors installed on a smartphone and processing same.

### BACKGROUND OF THE INVENTION

Indoor fires may break overnight and spread quickly. An early detection of the fire source is important in overcoming them effectively. Standard fire detectors are not always installed in every room and some of them tend to issue false alarms.

People use smartphones continuously and tend to place them near their bed when they go to sleep. It would be advantageous therefore to find a way to use one or more of the sensors on the smartphone and configure them in such a way that may provide the ability to sense and detect early signs of an indoor fire.

Even beyond the use of smartphones, it would be also advantageous to find another types of sensors which can be more reliable and configure them is such a way that may provide the ability to sense and detect early signs of an indoor fire.

### SUMMARY OF THE INVENTION

In order to address the aforementioned challenge, it has been suggested by the inventor of the present invention, to utilize the light detection and ranging (LiDAR) and/or Time-of-Flight (ToF) sensor installed on the smartphone, for sensing fire. These sensors are originally used for determining distance from objects. According to some embodiments of the present invention, these sensors are re-configured to detect a level of particles in the vicinity of the sleeping person and deduce, using software application running on the processor of the smartphone, and based on the sensed level of particle, whether a fire has broken. Once a fire has been detected, an alert can be issued either on the smartphone or to a remote server for immediate attention.

In order to address the aforementioned challenge, it has been also suggested by the inventor of the present invention, to utilize the light detection and ranging (LiDAR) and/or Time-of-Flight (ToF) sensors for sensing fire on standalone devices, not as a part of a smartphone.

The LiDAR and the ToF sensors are originally used for determining distance from objects. According to some embodiments of the present invention, these sensors may be re-configured to detect a level of particles in a room where they are placed and deduce, using software application running a processor, and based on the sensed level of particle, whether a fire has broken. Once a fire has been detected, an alert can be issued either by a device holding the sensor or to a remote server for immediate attention.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1A is a high-level block diagram illustrating a system for detecting a fire using a smartphone in accordance with embodiments of the present invention;

FIG. 1B is a high-level block diagram illustrating a system for detecting a fire using a standalone device in accordance with embodiments of the present invention;

FIG. 2A is a high-level flowchart illustrating a method for detecting a fire using a smartphone in accordance with embodiments of the present invention; and

FIG. 2B is a high-level flowchart illustrating a method for detecting a fire using a standalone device in accordance with embodiments of the present invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE INVENTION

Prior to setting forth the detailed description of the invention, it may be helpful to provide herein definitions of certain terms that will be used hereinafter.

The term “mobile communication device” as used herein is defined as any electronic device which is small enough to be carried by a human user and further has radio frequency (RF) wireless communication capabilities. And further can be placed in a stationary position. The mobile communication device is typically a phone such as a smartphone, a tablet, or a game console. The mobile communication device may support audio calls or transmitting and receiving data and may or not have a touch screen.

The term “LiDAR” is an acronym for “light detection and ranging”. As used herein it refers to any laser technology used to measure distances and map objects. A LiDAR sensor installed on a mobile communication device typically emits light pulses toward an object and captures the refracted light to create accurate 2D and 3D maps and determine the distance and size of the object by a dedicated LiDAR software application installed on the smartphone.

The term time-of-flight camera (ToF camera), also known as time-of-flight sensor (ToF sensor), as used herein is defined as a range imaging camera system for measuring distances between the camera and the subject for each point of the image based on time-of-flight, the round trip time of an artificial light signal, as provided by a laser or an LED. These sensors are installed in some smartphones. For all purposes, the terms ToF sensor and LiDAR sensor are used herein interchangeably.

The terms “memory” and “computer-readable storage medium” as used herein may refer to multiple structures, such as a plurality of memories or computer-readable storage mediums located within a mobile communication device or at a remote location. Additionally, one or more computer-readable storage mediums can be utilized in implementing a computer-implemented method. The term “computer-readable storage medium” should be understood to include tangible items and exclude carrier waves and transient signals.

The term “non-transitory computer-readable storage medium” as used herein, refers to any type of physical memory on which information or data readable by at least one processor can be stored. Examples include random access memory (RAM), read-only memory (ROM), volatile memory, nonvolatile memory, hard drives, CD ROMs, DVDs, flash drives, disks, any other optical data storage medium, a PROM, an EPROM, a FLASH-EPROM or any other flash memory, NVRAM, a cache, a register, any other memory chip or cartridge, and networked versions of the same.

The term “processor” or “processing device” as used herein may include at least one processor configured to execute computer programs, applications, methods, processes, or other software to perform embodiments described in the present disclosure. For example, a processing device may include one or more integrated circuits, microchips, microcontrollers, microprocessors, all or part of a central processing unit (CPU), graphics processing unit (GPU), or other circuits suitable for executing instructions or performing logic operations. The processing device may include at least one processor configured to perform functions of the disclosed methods. The processing device may include a single-core or multiple-core processor executing parallel processes simultaneously. The processing device may be implemented using a virtual machine architecture or other methods that provide the ability to execute, control, run, manipulate, store, etc., multiple software processes, applications, programs, etc. In another example, the processing device may include a multiple-core processor architecture (e.g., dual, quad-core, etc.) configured to provide parallel processing functionalities to allow a device associated with the processing device to execute multiple processes simultaneously. It is appreciated that other types of processor architectures could be implemented to provide the capabilities disclosed herein.

The term “non-transitory computer-readable storage medium” as used herein, refers to any type of physical memory on which information or data readable by at least one processor can be stored. Examples include random access memory (RAM), read-only memory (ROM), volatile memory, nonvolatile memory, hard drives, CD ROMs, DVDs, flash drives, disks, any other optical data storage medium, a PROM, an EPROM, a FLASH-EPROM or any other flash memory, NVRAM, a cache, a register, any other memory chip or cartridge, and networked versions of the same.

In the following description, various aspects of the present invention will be described. For purposes of explanation, specific configurations and details are set forth in order to provide a thorough understanding of the present invention. However, it will also be apparent to one skilled in the art that the present invention may be practiced without the specific details presented herein. Furthermore, well-known features may be omitted or simplified in order not to obscure the present invention.

Unless specifically stated otherwise, as apparent from the following discussions, it is appreciated that throughout the specification discussions utilizing terms such as “processing”, “computing”, “calculating”, “determining”, or the like, refer to the action and/or processes of a computer or computing system, or similar electronic computing device, that manipulates and/or transforms data represented as physical, such as electronic, quantities within the computing system's registers and/or memories into other data similarly represented as physical quantities within the computing system's memories, registers or other such information storage, transmission or display devices.

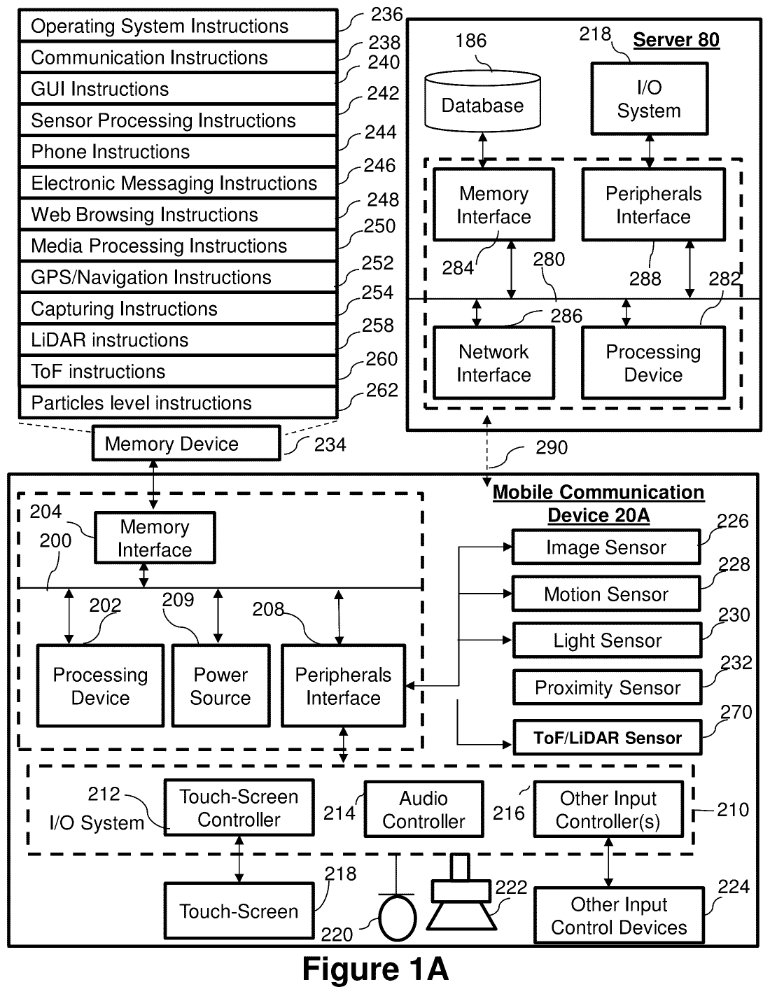

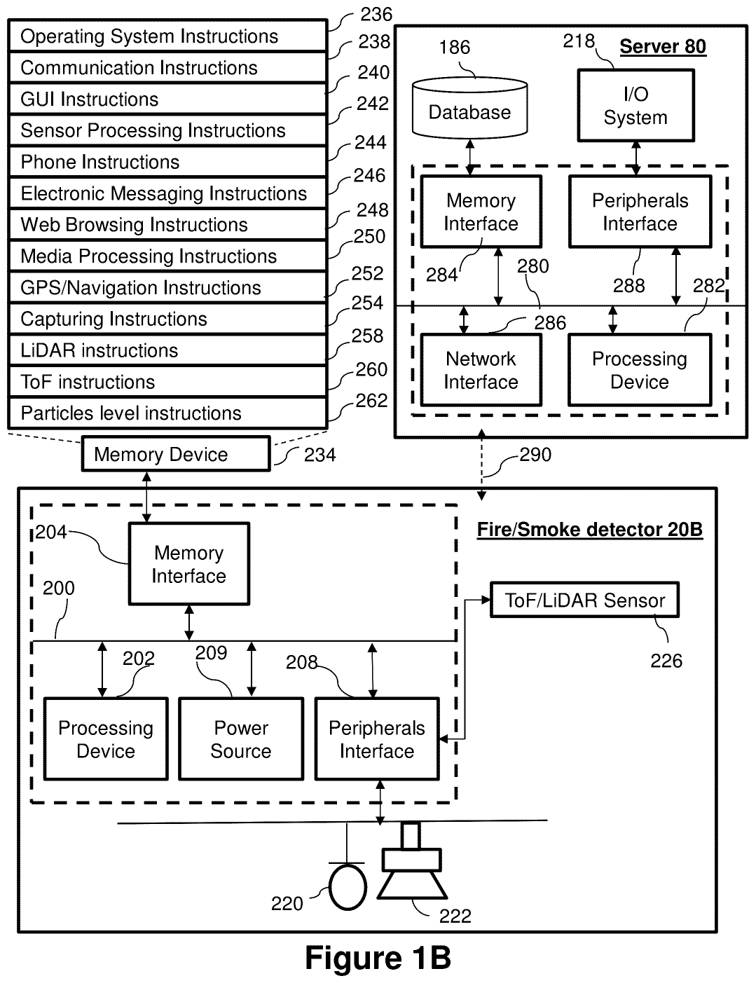

FIG. 1A shows a block diagram of the configuration of a mobile communication device 20A and a server 80 to serve as a fire alarm system based on LiDAR/ToF sensor technology. With regard to the mobile communication device 20A, and according to some embodiments, the mobile communication device 20A, directly or indirectly, may access a bus 200 (or another data transfer mechanism) that interconnects subsystems and components for transferring information within the mobile communication device 20A. For example, bus 200 may interconnect a processing device 202, a memory interface 204, and a peripherals interface 208 connected to an I/O system 210. Power source 209 provides the power to the mobile communication device and it may include a primary or a rechargeable battery (not shown), DC-DC converters (not shown) and other components required for the proper operation mobile communication device 20A.

In some embodiments, processing device 202 may use a memory interface 204 to access data and a software product stored on a memory device 234 or a non-transitory computer-readable medium device 234.

According to some embodiments, the peripherals interface 208 may also be connected to sensors, devices, and subsystems to facilitate multiple functionalities. In one embodiment, the peripherals interface 208 may be connected to an I/O system 210 configured to receive signals or input from devices and to provide signals or output to one or more devices that allow data to be received and/or transmitted by the mobile communication device 20. In one example, the I/O system 210 may include a touch screen controller 212, audio controller 214, and/or other types of input controller(s) 216. The touch screen controller 212 may be coupled to a touch screen 218. The touch screen 218 and the touch screen controller 212 may, for example, detect contact, and movement, using any of a plurality of touch sensitivity technologies, including but not limited to capacitive, resistive, infrared, and surface acoustic wave technologies as well as other proximity sensor arrays or other elements for determining one or more points of contact with the touch screen 218. The touch screen 218 may also, for example, be used to implement virtual or soft buttons and/or a keyboard. While a touch screen 218 is shown in FIG. 1, I/O system 210 may include a display screen (e.g., LCD or LED) in place of a touch screen 218.

According to some embodiments, the peripherals interface 208 may also be connected to an image sensor 226, a motion sensor 228, a light sensor 230, and/or a proximity sensor 232 to facilitate image capturing, orientation, lighting, and proximity functions. In addition, a GPS receiver may also be integrated with, or connected to, the mobile communication device 20, such as GPS receivers typically integrated into mobile communications devices. Alternatively, GPS software may permit the mobile communication device 20 to access an external GPS receiver (e.g., connecting via a serial port or Bluetooth).

Consistent with the present disclosure, the mobile communication device 20A may use a memory interface 204 to access a memory device 234. The memory device 234 may store an operating system 236, such as Android, iOS, MS Windows, Linux, or any other embedded operating system. Operating system 236 may include instructions for handling basic system services and for performing hardware-dependent tasks. In some implementations, the operating system 236 may be a kernel (e.g., Linux kernel).

The memory device 234 may also store communication instructions 238 to facilitate communicating with one or more additional devices, one or more computers, and/or one or more servers. The memory device 234 may include: graphical user interface instructions 240 to facilitate graphic user interface processing; sensor processing instructions 242 to facilitate sensor-related processing and functions; phone instructions 244 to facilitate phone-related processes and functions; electronic messaging instructions 246 to facilitate electronic-messaging-related processes and functions; web browsing instructions 248 to facilitate web browsing-related processes and functions; media processing instructions 250 to facilitate media processing-related processes and functions; GPS/navigation instructions 252 to facilitate GPS and navigation-related processes and instructions; capturing instructions 254 to facilitate processes and functions related to image sensor 226.

Each of the above-identified instructions and applications may correspond to a set of instructions for performing one or more functions described above. These instructions do not necessarily need to be implemented as separate software programs, procedures, or modules. The memory device 234 may include additional instructions or fewer instructions. Furthermore, various functions of the mobile communication device 20A may be implemented in hardware and/or software, including in one or more signal processing and/or application-specific integrated circuits.

Still referring to FIG. 1A, and according to some embodiments of the present invention, a server 80 for monitoring fires detected by on at least one mobile communication device 20A within an indoor space, is illustrated herein.

Processing device 282 may include at least one processor configured to execute computer programs, applications, methods, processes, or other software to perform embodiments described in the present disclosure.

In some embodiments, processing device 282 may use a memory interface 284 to access data and a software product stored on a memory device or a non-transitory computer-readable medium or to access a data structure 186.

According to some embodiments, the network interface 286 may provide two-way data communication to a network. In FIG. 1, communication 290 between the mobile communication device 20A and server 80 is represented by a dashed arrow. In one embodiment, the network interface 286 may include an integrated services digital network (ISDN) card, cellular modem, satellite modem, or a modem to provide a data communication connection over the Internet. As another example, the network interface 286 may include a wireless local area network (WLAN) card. In another embodiment, the network interface 286 may include an Ethernet port connected to radio frequency receivers and transmitters. The specific design and implementation of the network interface 286 may depend on the communications network(s) over which the mobile communication device 20A and the server 80 may communicate.

According to some embodiments, server 80 may also include a peripherals interface 288 coupled to the bus 280. The peripherals interface 288 may also be connected to devices, and subsystems to facilitate multiple functionalities as performed by the server 80. In some embodiments, those devices and subsystems may comprise a display screen (e.g., CRT or LCD) a USB port, and the like.

The components and arrangements shown in FIG. 1 for both server 80 and the mobile communication device 20A are not intended to limit the disclosed embodiments. As will be appreciated by a person skilled in the art having the benefit of this disclosure, numerous variations and/or modifications may be made to the depicted configuration of server 80 and the mobile communication device 20A. For example, not all the depicted components may be essential for the operation of server 80 or the mobile communication device 20A in all cases. Any component may be located in any appropriate part of server 80, and the components may be rearranged into a variety of configurations while providing the functionality of the disclosed embodiments. For example, some types of mobile communication devices 20A may include only part of the depicted sensors and include sensors which are not depicted.

Mobile communication device 20A may include Lidar/ToF sensor 270. Lidar instructions 258 and ToF instructions 260 are computer code instructions stored on memory device 234 and when executed, cause processing device 202, to control Lidar/ToF sensor 270 to emit a light beam and receive reflections and to calculate range, distance and 3D mapping of the scene and the like.

According to some embodiments of the present invention, memory device 234 may further include particles level instructions 262 which cause processing device 202, when executed, to monitor level of reflections to Lidar/ToF sensor 270 over a plurality of ranges measured from Lidar/ToF sensor 270 and assess the level of air particles in the surrounding.

This is done by splitting the line of sight of Lidar/ToF sensor 270 into a plurality of segments (e.g., 20 cm each) and monitoring the level of reflections from each segment. A higher density of air particles in one segment shall indicate a higher level of air particles compared with a lower level of reflection in a different segment.

As smoke particles tend to go up it is suggested by inventor of the present invention to have the smartphone placed on a surface such as a drawer near bed with the Lidar/ToF sensor 270 facing up towards the ceiling. This usually means that the screen will be face down. Thus, the Lidar/ToF sensor 270 will be able to detect particles at a range of around 2 m and beyond which is closer to the ceiling which forms a full barrier to Lidar/ToF sensor 270. In such an arrangement, a substantially higher density of particles at the segments 2 m-2.5 m (with standard ceiling) along the line of sight of Lidar/ToF sensor 270 may ne indicative of smoke and thus invoke a fire alarm.

The above can be applied with a standalone device not implemented by a smartphone and the detector should be placed similarly, facing the ceiling so as to be able to detect higher density of air particles closer to the ceiling.

Calibration data retrieved over many experiments may show the correlation and other statistical relationship between certain levels of reflections and level of air particles. The calibration data may be in the form of a model which may be used in order to convert a level of reflections in one or more of the segments into a level of air particles above a predefined size, or particles which resemble smoke. particles level instructions 262 may apply calibration data to the readings received from Lidar/ToF sensor 270 and deduce the level of particles in the surrounding of mobile communication device 20.

Other sensors such as proximity sensor 232 may be used to guarantee that mobile communication device 20 is placed in a stationary location and with the Lidar/ToF sensor 270 exposed to the scene (e.g., not facing an obstacle). Additionally, the light sensor 230 and image sensor 226 can also be used to verify an event of fire, once smoke has been detected based on the level of air particles.

Once smoke and/or file have been detected, an alert such as an alarm may be issued at mobile communication device 20. Alternatively, and additionally, the alert may be transmitted to server 80 together with other data such as the location of mobile communication device 20 and possibly further identification data relating to the user of mobile communication device 20.

In order to guarantee normal operation of the smartphone as smoke detector, the mobile communication device 20 may inform the user of any deficiency in setting it up, such as lack of battery or an obstructed Lidar/ToF sensor 270.

According to some embodiments of the present invention, the processing device is further configured to issue an alert as soon as it detects smoke. According to some embodiments, the alert is sent to a server in communication with the mobile communication device.

According to some embodiments of the present invention, the mobile communication device further comprises a proximity sensor and wherein the processing device is further configured to alert that the mobile communication device is placed such that the LiDAR/TOF sensor is obstructed.

According to some embodiments of the present invention, the processing device maps the reflection measurements onto level of air particles by measuring reflections in various segments along a line of sight of the LiDAR/TOF sensor, wherein level of reflections along the line of sight are represented by a histogram. The segments may be 10 cm to 20 cm each and smoke is determined when a level of particles is significantly higher in segments of the histogram at a range of approximately 2 m-3 m from the LiDAR/TOF sensor.

FIG. 1B shows a block diagram of the configuration of a standalone device 20B and a server 80 to serve as a fire alarm system based on LiDAR/ToF sensor technology. With regard to the standalone device 20B, and according to some embodiments, the standalone device 20B, directly or indirectly, may access a bus 200 (or another data transfer mechanism) that interconnects subsystems and components for transferring information within the standalone device 20B. For example, bus 200 may interconnect a processing device 202, a memory interface 204, and a peripherals interface 208 connected to an I/O system 210. Power source 209 provides the power to the standalone device and it may include a primary or a rechargeable battery (not shown), DC-DC converters (not shown) and other components required for the proper operation of the standalone device 20B.

In some embodiments, processing device 202 may use a memory interface 204 to access data and a software product stored on a memory device 234 or a non-transitory computer-readable medium device 234.

According to some embodiments, the peripherals interface 208 may also be connected to sensors, devices, and subsystems to facilitate multiple functionalities. In one embodiment, the peripherals interface 208 may be connected to an I/O system 210 configured to receive signals or input from devices and to provide signals or output to one or more devices that allow data to be received and/or transmitted by the standalone device 20B. In one example, the I/O system 210 may include a touch screen controller 212, audio controller 214, and/or other types of input controller(s) 216. The touch screen controller 212 may be coupled to a touch screen 218. The touch screen 218 and the touch screen controller 212 may, for example, detect contact, and movement, using any of a plurality of touch sensitivity technologies, including but not limited to capacitive, resistive, infrared, and surface acoustic wave technologies as well as other proximity sensor arrays or other elements for determining one or more points of contact with the touch screen 218. The touch screen 218 may also, for example, be used to implement virtual or soft buttons and/or a keyboard. While a touch screen 218 is shown in FIG. 1B, I/O system 210 may include a display screen (e.g., LCD or LED) in place of a touch screen 218.

According to some embodiments, the peripherals interface 208 may also be connected to an image sensor 226, a motion sensor 228, a light sensor 230, and/or a proximity sensor 232 to facilitate image capturing, orientation, lighting, and proximity functions. In addition, a GPS receiver may also be integrated with, or connected to, the standalone device 20, such as GPS receivers typically integrated into mobile communications devices. Alternatively, GPS software may permit the standalone device 20B to access an external GPS receiver (e.g., connecting via a serial port or Bluetooth).

Consistent with the present disclosure, standalone device 20B may use a memory interface 204 to access a memory device 234. The memory device 234 may store an operating system 236, such as Android, iOS, MS Windows, Linux, or any other embedded operating system. Operating system 236 may include instructions for handling basic system services and for performing hardware-dependent tasks. In some implementations, the operating system 236 may be a kernel (e.g., Linux kernel).

The memory device 234 may also store communication instructions 238 to facilitate communicating with one or more additional devices, one or more computers, and/or one or more servers. The memory device 234 may include: graphical user interface instructions 240 to facilitate graphic user interface processing; sensor processing instructions 242 to facilitate sensor-related processing and functions; phone instructions 244 to facilitate phone-related processes and functions; electronic messaging instructions 246 to facilitate electronic-messaging-related processes and functions; web browsing instructions 248 to facilitate web browsing-related processes and functions; media processing instructions 250 to facilitate media processing-related processes and functions; GPS/navigation instructions 252 to facilitate GPS and navigation-related processes and instructions; capturing instructions 254 to facilitate processes and functions related to image sensor 226.

Each of the above-identified instructions and applications may correspond to a set of instructions for performing one or more functions described above. These instructions do not necessarily need to be implemented as separate software programs, procedures, or modules. The memory device 234 may include additional instructions or fewer instructions. Furthermore, various functions of the standalone device 20B may be implemented in hardware and/or software, including in one or more signal processing and/or application-specific integrated circuits.

Still referring to FIG. 1B, and according to some embodiments of the present invention, a server 80 for monitoring fires detected by on at least one standalone device 20B within an indoor space, is illustrated herein.

Processing device 282 may include at least one processor configured to execute computer programs, applications, methods, processes, or other software to perform embodiments described in the present disclosure.

In some embodiments, processing device 282 may use a memory interface 284 to access data and a software product stored on a memory device or a non-transitory computer-readable medium or to access a data structure 186.

According to some embodiments, the network interface 286 may provide two-way data communication to a network. In FIG. 1B, communication 290 between the standalone device 20B and server 80 is represented by a dashed arrow. In one embodiment, the network interface 286 may include an integrated services digital network (ISDN) card, cellular modem, satellite modem, or a modem to provide a data communication connection over the Internet. As another example, the network interface 286 may include a wireless local area network (WLAN) card. In another embodiment, the network interface 286 may include an Ethernet port connected to radio frequency receivers and transmitters. The specific design and implementation of the network interface 286 may depend on the communications network(s) over which the standalone device 20 and the server 80 may communicate.

According to some embodiments, the server 80 may also include a peripherals interface 288 coupled to the bus 280. The peripherals interface 288 may also be connected to devices, and subsystems to facilitate multiple functionalities as performed by the server 80. In some embodiments, those devices and subsystems may comprise a display screen (e.g., CRT or LCD) a USB port, and the like.

The components and arrangements shown in FIG. 1B for both server 80 and the standalone device 20 are not intended to limit the disclosed embodiments. As will be appreciated by a person skilled in the art having the benefit of this disclosure, numerous variations and/or modifications may be made to the depicted configuration of server 80 and the standalone device 20B. For example, not all the depicted components may be essential for the operation of server 80 or the standalone device 20B in all cases. Any component may be located in any appropriate part of server 80, and the components may be rearranged into a variety of configurations while providing the functionality of the disclosed embodiments. For example, some types of standalone devices 20 may include only part of the depicted sensors and include sensors which are not depicted.

Standalone device 20B may include Lidar/ToF sensor 270. Lidar instructions 258 and ToF instructions 260 are computer code instructions stored on memory device 234 and when executed, cause processing device 202, to control Lidar/ToF sensor 270 to emit a light beam and receive reflections and to calculate range, distance and 3D mapping of the scene, as the case may be.

According to some embodiments of the present invention, memory device 234 may further include particles level instructions 262 which cause processing device 202, when executed, to monitor level of reflections to Lidar/ToF sensor 270 over a plurality of ranges and assess the level of air particles in the surrounding.

This is done by splitting the line of sight of Lidar/ToF sensor 270 into a plurality of segments and monitoring the level of reflections from each segment. A higher density of air particles in one segment shall indicate a higher level of air particles compared with a lower level of reflections in a different segment.

Calibration data retrieved over many experiments may show the correlation and other statistical relationship between certain levels of reflections and level of air particles. The calibration data may be in the form of a model which may be used in order to convert a level of reflections in one or more of the segments into a level of air particles above a predefined size, or particles which resemble smoke. particles level instructions 262 may apply calibration data to the readings received from Lidar/ToF sensor 270 and deduce the level of particles in the surrounding of standalone device 20B.

Other sensors such as proximity sensor 232 may be used to guarantee that standalone device 20B is places in a stationary location and with the Lidar/ToF sensor 270 exposed to the scene (e.g., not facing an obstacle). Additionally, the light sensor 230 and image sensor 226 can also be used to verify an event of fire, once smoke has been detected based on the level of air particles.

Once smoke and/or file have been detected, a alert such as an alarm may be issued at standalone device 20. Alternatively, and additionally, the alert may be transmitted to server 80 together with other data such as the location of standalone device 20 and possibly further identification data relating to the user of standalone device 20.

In order to guarantee normal operation of the standalone device 20B as a smoke detector, the standalone device 20 may inform the user of any deficiency in setting it up, such as lack of battery or an obstructed Lidar/ToF sensor 270.

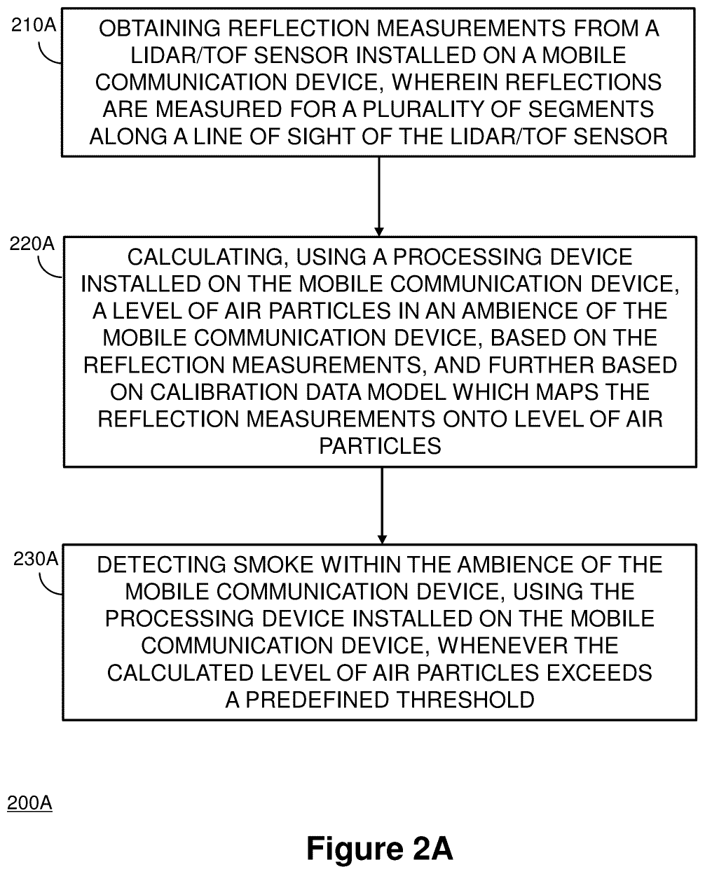

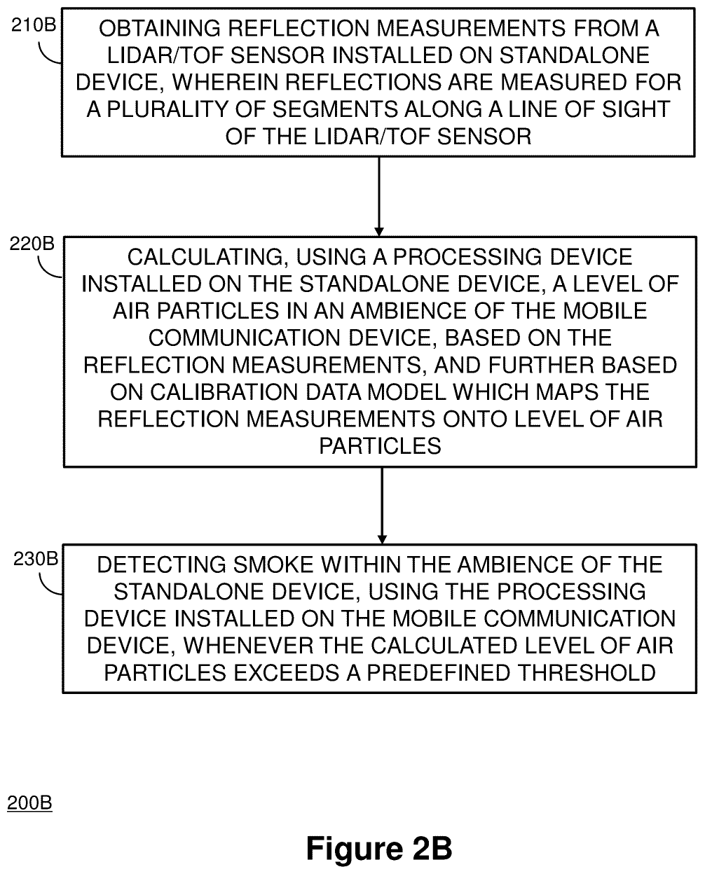

FIG. 2A depicts a high-level flowchart illustrating method 200A for detecting a fire via a smartphone. Method 200A is a method of early detection of an indoor fire using a mobile communication device having a light detection and ranging (LiDAR) sensor/Time of Flight (TOF) sensor. Method 200A may include the following steps: obtaining reflection measurements from the LiDAR/TOF sensor, wherein reflections are measured for a plurality of segments along a line of sight of the LiDAR/TOF sensor 210A; calculating, using a processing device installed on the mobile communication device, a level of air particles in an ambience of the mobile communication device, based on the reflection measurements, and further based on calibration data model which maps the reflection measurements onto level of air particles 220A; and detecting smoke within the ambience of the mobile communication device, using the processing device installed on the mobile communication device, whenever the calculated level of air particles goes beyond a predefined threshold 230A.

FIG. 2B depicts a high-level flowchart illustrating a method 200B for detecting a fire via a standalone device. Method 200B is a method of early detection of an indoor fire using a standalone device having a light detection and ranging (LiDAR) sensor/Time of Flight (TOF) sensor. Method 200B may include the following steps: obtaining reflection measurements from the LiDAR/TOF sensor, wherein reflections are measured for a plurality of segments along a line of sight of the LiDAR/TOF sensor 210B; calculating, using a processing device installed on the standalone device, a level of air particles in an ambience of the standalone device, based on the reflection measurements, and further based on calibration data model which maps the reflection measurements onto level of air particles 220B; and detecting smoke within the ambience of the standalone device, using the processing device installed on the standalone device, whenever the calculated level of air particles goes beyond a predefined threshold 230B.

It is further understood that some embodiments of the present invention may be embodied in the form of a system, a method, or a computer program product. Similarly, some embodiments may be embodied as hardware, software, or a combination of both. Some embodiments may be embodied as a computer program product saved on one or more non-transitory computer-readable medium (or mediums) in the form of computer-readable program code embodied thereon. Such non-transitory computer-readable medium may include instructions that when executed cause a processor to execute method steps in accordance with embodiments. In some embodiments, the instructions stored on the computer-readable medium may be in the form of an installed application and in the form of an installation package.

1. A system for early detection of an indoor fire using a mobile communication device having a light detection and ranging (LiDAR) sensor or a Time of Flight (TOF) sensor, the system comprising:
a processing device installed on the mobile communication device;
a memory device installed on the mobile communication device storing a set of instructions that, when executed, cause the processing device to:
obtain reflection measurements from the LiDAR/TOF sensor, wherein reflections are measured for a plurality of segments along a line of sight of the LiDAR/TOF sensor;
calculate a level of air particles in an ambience of the mobile communication device, based on the reflection measurements, and further based on calibration data model which maps the reflection measurements onto level of air particles; and
detect smoke within the ambience of the mobile communication device, whenever the calculated level of air particles goes beyond a predefined threshold.

2. The system according to claim 1, the processing device is further configured to issue an alert as soon as it detects smoke.

3. The system according to claim 2, wherein the alert is sent to a server in communication with the mobile communication device.

4. The system according to claim 1, wherein the mobile communication device further comprises a proximity sensor and wherein the processing device is further configured to alert that the mobile communication device is placed such that the LiDAR/TOF sensor is obstructed.

5. The system according to claim 1, the processing device maps the reflection measurements onto level of air particles by measuring reflections in various segments along a line of sight of the LiDAR/TOF sensor, wherein level of reflections along the line of sight are represented by a histogram.

6. The system according to claim 5, wherein the segments are 10 cm to 20 cm each.

7. The system according to claim 6, wherein smoke is determined when a level of particles is significantly higher in segments of the histogram at a range of approximately 2 m-3 m from the LiDAR/TOF sensor.

8. A method of early detection of an indoor fire using a mobile communication device having a light detection and ranging (LiDAR) sensor/Time of Flight (TOF) sensor, the method comprising:
obtaining reflection measurements from the LiDAR/TOF sensor, wherein reflections are measured for a plurality of segments along a line of sight of the LiDAR/TOF sensor;
calculating, using a processing device installed on the mobile communication device, a level of air particles in an ambience of the mobile communication device, based on the reflection measurements, and further based on calibration data model which maps the reflection measurements onto level of air particles; and
detecting smoke within the ambience of the mobile communication device, using the processing device installed on the mobile communication device, whenever the calculated level of air particles goes beyond a predefined threshold.

9. The method according to claim 8, wherein the processing device is further configured to issue an alert as soon as it detects smoke.

10. The method according to claim 9, wherein the alert is sent to a server in communication with the mobile communication device.

11. The method according to claim 8, wherein the mobile communication device further comprises a proximity sensor and wherein the processing device is further configured to alert that the mobile communication device is placed such that the LiDAR/TOF sensor is obstructed.

12. The method according to claim 8, wherein the processing device maps the reflection measurements onto level of air particles by measuring reflections in various segments along a line of sight of the LiDAR/TOF sensor, wherein level of reflections along the line of sight are represented by a histogram.

13. The method according to claim 12, wherein the segments are 10 cm to 20 cm each.

14. The method according to claim 13, wherein smoke is determined when a level of particles is significantly higher in segments of the histogram at a range of approximately 2 m-3 m from the LiDAR/TOF sensor.

15. A system for early detection of an indoor fire using a standalone device having a light detection and ranging (LiDAR) sensor/Time of Flight (TOF) sensor, the system comprising:
a processing device installed on the standalone device;
a memory device installed on the standalone device storing a set of instructions that, when executed, cause the processing device to:
obtain reflection measurements from the LiDAR/TOF sensor, wherein reflections are measured for a plurality of segments along a line of sight of the LiDAR/TOF sensor;
calculate a level of air particles in an ambience of the standalone device, based on the reflection measurements, and further based on calibration data model which maps the reflection measurements onto level of air particles; and
detect smoke within the ambience of the standalone device, whenever the calculated level of air particles exceeds a predefined threshold.

16. The system according to claim 15, the processing device is further configured to issue an alert as soon as it detects smoke.

17. The system according to claim 16, wherein the alert is sent to a server in communication with the standalone device.

18. The system according to claim 15, wherein the standalone device further comprises a proximity sensor and wherein the processing device is further configured to alert that the mobile communication device is placed such that the LiDAR/TOF sensor is obstructed.

19. The system according to claim 15, the processing device maps the reflection measurements onto level of air particles by measuring reflections in various segments along a line of sight of the LiDAR/TOF sensor, wherein level of reflections along the line of sight are represented by a histogram.

20. The system according to claim 19, wherein the segments are 10 cm to 20 cm each.
